(19) United States
(12) Patent Application Publication
(10) Pub. No.: US 2026/0079083 A1
(43) Pub. Date: Mar. 19, 2026

(54) METHOD AND DEVICE FOR DUNE SAMPLING AND SHORELINE MODELING

(71) Applicant: United States of America as Represented by The Secretary of The Army, Alexandria, VA (US)

(72) Inventors: Lee Ann Provost, Vicksburg, MS (US); Duncan B. Bryant, Vicksburg, MS (US); Timothy Hynum, Vicksburg, MS (US)

(73) Assignee: United States of America as Represented by The Secretary of The Army, Alexandria, VA (US)

(21) Appl. No.: 18/885,654

(22) Filed: Sep. 14, 2024

(51) Int. Cl. G01N 1/12; G01N 33/24

(52) U.S. Cl. CPC G01N 1/12; G01N 33/24

(57) ABSTRACT

In one embodiment, an extraction box includes a bottom, a right side wall, and a left side wall. A first wall forms a back wall on a back side of the extraction box. A second wall forms a front wall on a front side of the extraction box. The second wall is movable between a closed position to enclose the front side and an open position to open up the front side of the extraction box. A removable connector is removably attached to the right side wall and the left side wall to be disposed adjacent a top side of the extraction box, to be driven in a direction between the front side and the back side of the extraction box and to be lifted in an upward direction. The removable connector is removable from the extraction box to provide an open top side of the extraction box.

### STATEMENT OF GOVERNMENT INTEREST

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees.

### CROSS-REFERENCE TO RELATED APPLICATIONS

### BACKGROUND

### Field of the Invention

The present invention relates to dune restoration and, more specifically, to apparatuses and methods for extracting a dune sample in its natural state and testing the extracted dune sample in a controlled environment for investigating dune stability.

### Description of the Related Art

This section introduces aspects that may help facilitate a better understanding of the invention. Accordingly, the statements of this section are to be read in this light and are not to be understood as admissions about what is prior art or what is not prior art.

Conventional dune stability evaluation relies on physical models utilizing mimic vegetation, or transplanted vegetation grown for a limited amount of time but are not representative of a naturally established system. This is a disadvantage as there are ecological components within a natural system that are not represented in those experiments which may contribute to dune stability but have not been measured.

### SUMMARY

To mitigate uncertainty in physical modeling of dune stability due to vegetation, there is a need to obtain live samples that are representative of a dune in its natural state rather than relying on constructed mimics. A dune sampler is configured to extract an intact dune segment for transport and testing in a laboratory flume.

In embodiments, the dune sampler is a steel box designed to sample a coastal dune segment with vegetation intact for laboratory flume testing. The box is designed to connect to heavy machinery, to allow sampling of a coastal dune, with removable walls to allow wave propagation into or wave overtopping through the box for testing. The box may be constructed of a steel floor, and two non-removable sides. The front contains vertical tracks and a guillotine-style drop gate, with a hit point or hammer point. The back of the box contains a bolted-on steel plate. The sampler is equipped with a removable connector to allow connection to a standard excavator or backhoe. It is designed so the vertical gate is held in the open position while the box is driven into the sample. The bottom of the gate, as well as the front edges of the box are tapered to allow smoother cutting into the sample. The gate can then be dropped and hit with a mallet or driver on a hit point to drop the gate to cut through sediment and vegetation and seal off the sample. After lifting and transporting the extraction box to a flume and placing the box into the flume for testing, the front gate and back plate can be removed to allow wave propagation into or through the box for testing. The excavator connector can be removed to allow full profile view of the sample for measurements during testing.

A unique feature of the dune sampler in the form of an extraction box is that it allows for the reconfiguration of the sampler to transition smoothly from a contained unit, for testing purposes. The dune sampler allows for direct testing within the sampler and does not require the removal of the sample from the sampler itself. This allows for ease and efficiency of testing and reduces the risk of altering the sample before testing.

According to an aspect of the present invention, an extraction box comprises a bottom, a right side wall connected to the bottom, and a left side wall connected to the bottom. A first wall is connected to the bottom, the right side wall, and the left side wall, to form a back wall on a back side of the extraction box. A second wall is connected to the bottom, the right side wall, and the left side wall, to form a front wall on a front side of the extraction box. The second wall is movable between a closed position to enclose the front side of the extraction box and an open position to open up the front side of the extraction box. A removable connector is removably attached to the right side wall and the left side wall to be disposed adjacent a top side of the extraction box, to be driven in a direction between the front side and the back side of the extraction box and to be lifted in an upward direction. The removable connector is removable from the extraction box to provide an open top side of the extraction box.

In accordance with another aspect, a dune sample extraction method comprises: holding an extraction box having a bottom; a right side wall connected to the bottom; a left side wall connected to the bottom; a first wall connected to the bottom, the right side wall, and the left side wall, to form a back wall on a back side of the extraction box; a second wall connected to the bottom, the right side wall, and the left side wall, to form a front wall on a front side of the extraction box; by a removable connector removably attached to the right side wall and the left side wall to be disposed adjacent a top side of the extraction box, the second wall being disposed in an open position to open up the front side of the extraction box; driving the open front side of the extraction box, through the removable connector, into a dune to collect a dune sample; moving the second wall from the open position to a closed position to cut through sediment and vegetation to separate the dune sample in the extraction box from the dune; lifting the extraction box from the dune by the removable connector; transporting the extraction box to a flume having two side walls extending in a longitudinal direction; lowering the extraction box into the flume to position the right side wall of the extraction box next to one side wall of the flume and the left side wall of the extraction box next to another side wall of the flume; and removing at least one of the first wall or the second wall from the extraction box to allow wave propagation into the extraction box for testing the dune sample.

In accordance with yet another aspect, a dune sample extraction method comprises: holding an extraction box having a bottom; a right side wall connected to the bottom; a left side wall connected to the bottom; a first wall connected to the bottom, the right side wall, and the left side wall, to form a back wall on a back side of the extraction box; a second wall connected to the bottom, the right side wall, and the left side wall, to form a front wall on a front side of the extraction box; by a removable connector removably attached to the right side wall and the left side wall to be disposed adjacent a top side of the extraction box, the second wall being disposed in an open position to open up the front side of the extraction box; driving the open front side of the extraction box, through the removable connector, into a dune to collect a dune sample; moving the second wall from the open position to a closed position to cut through sediment and vegetation to separate the dune sample in the extraction box from the dune; lifting the extraction box from the dune; and performing test on the dune sample in the extraction box without removing the dune sample from the extraction box.

### BRIEF DESCRIPTION OF THE DRAWINGS

Embodiments of the invention will become more fully apparent from the following detailed description, the appended claims, and the accompanying drawings in which like reference numerals identify similar or identical elements.

FIG. 1 is a perspective view of an example of a dune sampler in the form of an extraction box without the front wall and the back wall.

FIG. 2 is a top plan view of the extraction box.

FIG. 3A is a front elevational view of the extraction box.

FIG. 3B is a rear elevational view of the extraction box.

FIG. 4A is a side elevational view of the extraction box with the front wall in the closed position.

FIG. 4B is a side elevational view of the extraction box with the front wall in the open position.

FIG. 5 shows a backhoe connected to the removable connector of an extraction box for manipulating and maneuvering the extraction box hydraulically to extract a dune segment.

FIG. 6 shows the extraction box disposed in a flume for testing the extracted dune sample with vegetation intact.

FIG. 7 is a flow diagram illustrating an example of a dune segment extraction and testing process.

### DETAILED DESCRIPTION

Detailed illustrative embodiments of the present invention are disclosed herein. However, specific structural and functional details disclosed herein are merely representative for purposes of describing example embodiments of the present invention. The present invention may be embodied in many alternate forms and should not be construed as limited to only the embodiments set forth herein. Further, the terminology used herein is for the purpose of describing particular embodiments only and is not intended to be limiting of example embodiments of the invention.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

### 1. Introduction

In many locations along U.S. shorelines, coastal dune and beach systems are highly engineered for the purpose of protecting critical infrastructure, constraining erosion, supporting wildlife, and driving economic activity. While dune restoration has recently gained attention as a “green engineering” alternative to shoreline armoring and as a means to augment beach nourishment efforts, restored dunes are still significantly engineered, and the restoration process involves adding large volumes of sediment to the beach and manipulating that sediment with heavy machinery. This study is performed to understand how vegetation can reduce dune erosion and ultimately strengthen coastal dunes though less-invasive measures.

Coastal dune ecosystems are critically important for shoreline protection and significant resources have been allocated to their conservation and restoration. Dune vegetation is known to modify dune ecosystem response to wind, waves, and storms, but little focus has been given to understanding how vegetation, specifically belowground structures and associated microbial symbionts, can enhance dune stability. Further, coastal dune communities often support diverse vegetation assemblages and may have similarly diverse restoration histories. Little is known about how different species and previous management activities alter sediment strength, stability, and overall dune morphology. The dune component of a beach nourishment effort may provide the majority of the flood protection benefits. It remains unknown how dune characteristics such as the vegetation cover, the angle of repose, and previous restoration efforts alter dune resistance and resilience to storms.

To address these knowledge gaps, the researchers have built an idealized numerical dune model and simulated storm response under varied angles of repose and critical shear stresses, two variables modified by vegetation. The parameterization of these variables come from measurements of aboveground and belowground vegetation, dune slopes, and sediment properties. Dune erosion is simulated under different conditions using a physical model. A large-scale experiment is deployed to evaluate how modifying dune planting practices can increase the rate of dune establishment and enhance belowground biomass following restoration. By understanding how aboveground vegetation and belowground vegetation stabilize and bind sand, restoration efforts can become more targeted and resistant to future storms. Overall, this interdisciplinary study enhances understanding of dune resilience and erosion and how vegetation contributes to these metrics, ultimately providing the knowledge needed to improve upon coastal management strategies for flood-risk protection.

The researchers have performed a study to relate sand dune vegetation properties to dune stability metrics and erosion rates using field measurements, physical models, and idealized numerical models. By linking field data and models, they have improved the understanding of how dunes with different vegetation cover and restoration histories respond to extreme storm events. By coupling field surveys and experiments with physical and numerical models, the researchers have generated knowledge that will improve current dune management and restoration practices allowing for more optimized planning for flood risk protection.

Many current beach and dune management practices focus nearly exclusively on engineering design principles that do not explicitly include the effects of ecological interactions and processes in controlling beach/dune stability and geomorphic evolution. This study therefore aims to address a key aspect of this challenge by advancing understanding of how vegetation enhances dune resiliency and reduces dune erosion. Field and greenhouse experiments are conducted to evaluate different restoration approaches and test novel planting strategies. Field surveys are used to evaluate how the vegetation and sediment properties of previously restored dunes compare to natural dunes. The models are used to evaluate how vegetation, by altering the angle of repose and modifying sediment shear strength, can enhance dune stability. Collectively, the results can be used to better manage sand dunes and engineer more resilient and resistant dunes during restoration efforts.

### 2. Physical Model to Evaluate Dune Erosion

A physical model is used to evaluate the impact of belowground biomass on dune stability. A dune segment is extracted from a field site. A controlled laboratory experiment is implemented to investigate the physical role of ecological components on dune stability that cannot easily be replicated by dune model construction.

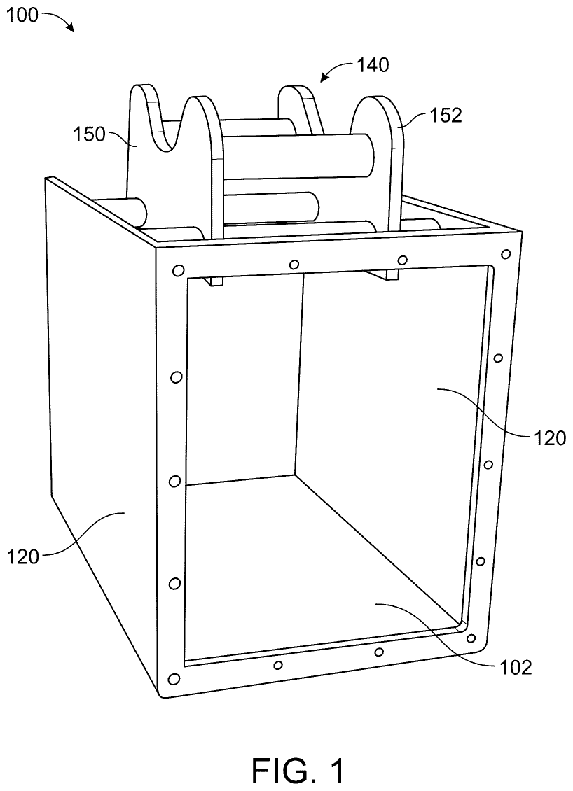

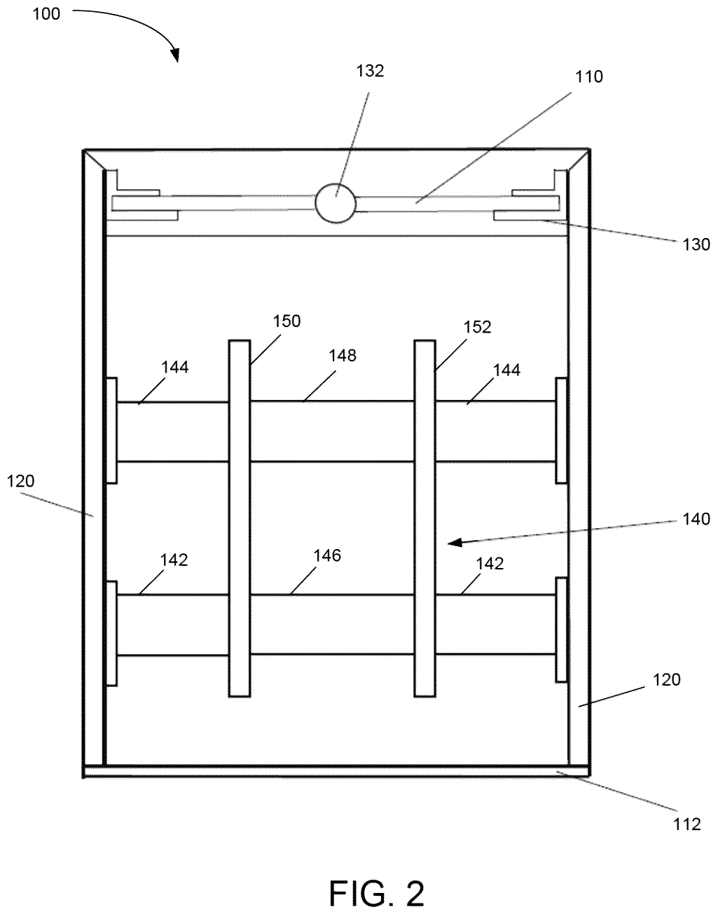

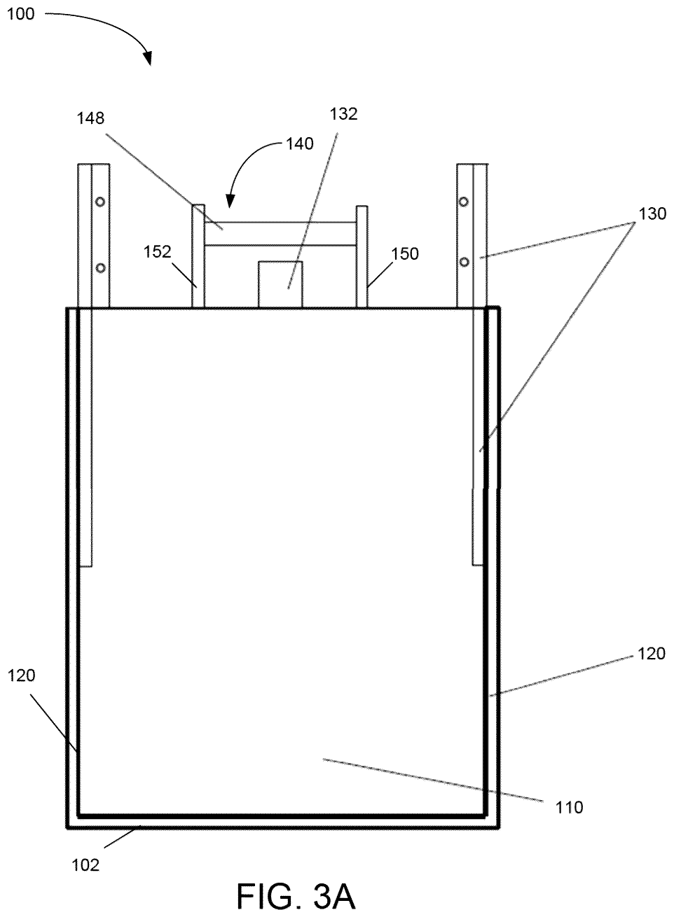

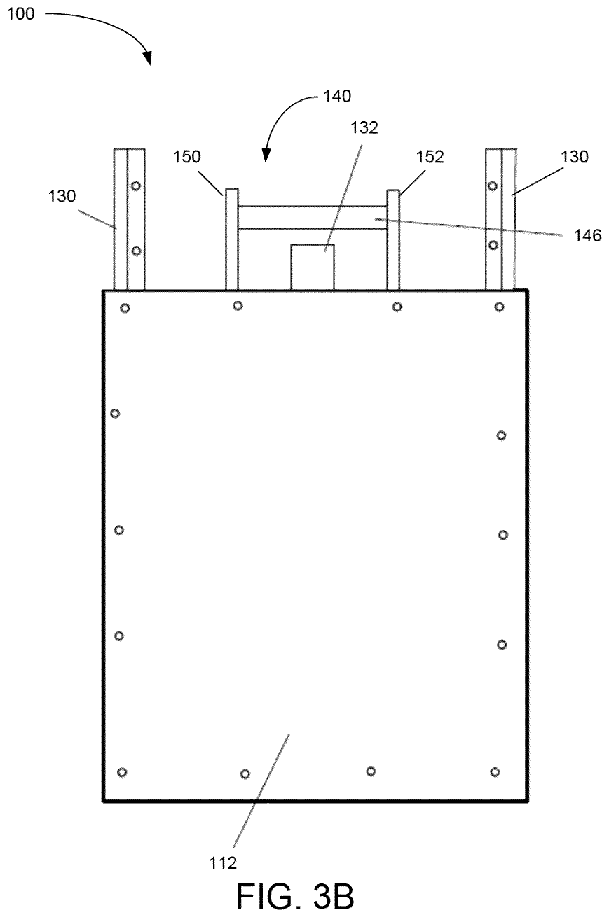

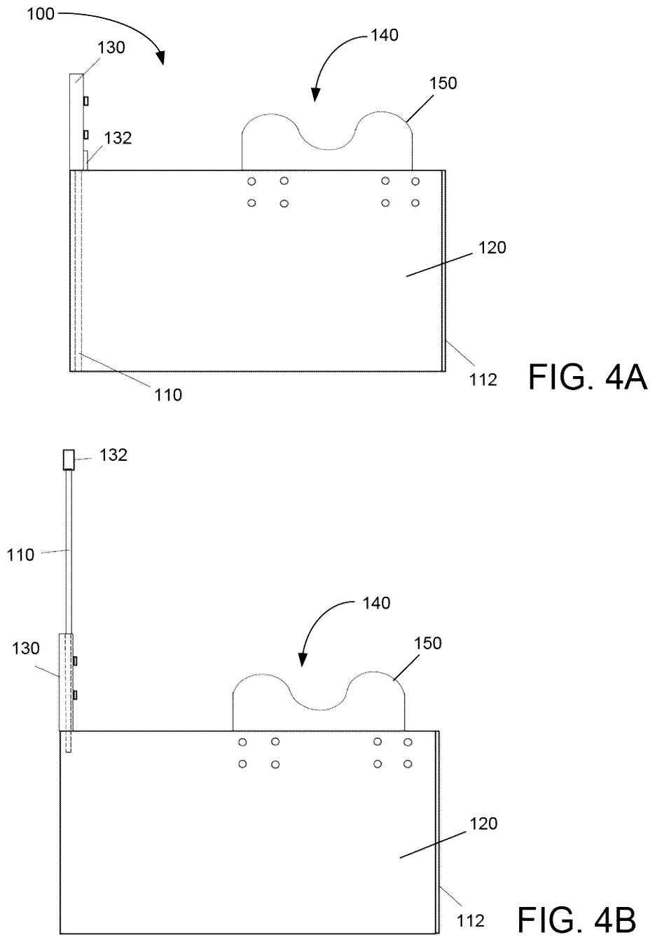

FIG. 1 is a perspective view of an example of a dune sampler in the form of an extraction box without the front wall and the back wall. FIG. 2 is a top plan view of the extraction box. FIG. 3A is a front elevational view of the extraction box. FIG. 3B is a rear elevational view of the extraction box. FIG. 4A is a side elevational view of the extraction box with the front wall in the closed position. FIG. 4B is a side elevational view of the extraction box with the front wall in the open position.

In this embodiment, the dune sampler is a metal box 100 designed to sample a coastal dune segment with vegetation intact for laboratory flume testing. The box is designed to connect to heavy machinery (e.g., an excavator or a backhoe), to allow sampling of a coastal dune, with removable walls (front wall 110 or second wall 110 and back wall 112 or first wall 112) to allow wave propagation into or through the box for testing. The box 100 may be constructed of a steel floor or bottom 102, and two non-removable steel sides or side walls 120. The bottom 102 and two side walls 120 form a sampler frame or box frame. The frame structure may be formed by casting or welding. The front side of the side walls 120 may include vertical tracks 130 and a guillotine-style drop gate as the front wall 110, with a hit point or hammer point 132. The front wall 110 is slidable or movable along the vertical tracks 130 between the open position to open the front side and the closed position to enclose the front side of the extraction box 100. The back of the box contains a bolted-on steel plate as the removable back plate 112. The dune sampler or extraction box 100 is equipped with a removable connector 140 to allow connection to a standard excavator or backhoe.

The extraction box 100 may be made of another metal or some other material. The material and dimensions selected should keep the box 100 sufficiently strong structurally to extract and hold the dune sample without damage or deformation. The bottom 102 and side walls 120 are sufficiently strong without the need for bracing or reinforcement structures. The use of bracing or reinforcement structures may interfere with dune sample extraction, for instance, by rendering it difficult to drive the extraction box 100 into a dune to extract a dune sample. It may also interfere with wave propagation in a flume or wave channel for testing the dune sample in the extraction box. In one example, the steel box is about 4 ft long×2.5 ft wide×3 ft deep with a thickness of about ¾ in. The dimensions may vary, for instance, by ±10% or more.

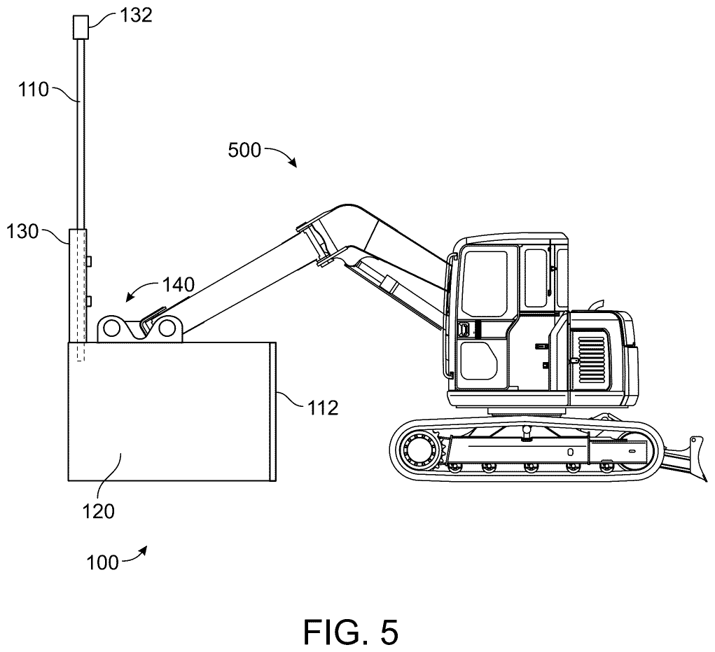

FIG. 5 shows a backhoe 500 connected to the removable connector 140 of an extraction box 100 for manipulating and maneuvering the extraction box hydraulically to extract a dune segment. The box 100 is configured to be driven into the dune from the side with the vertical front gate 110 held in the open position. The gate 110 can then be released from the tracks dropped first, and then hit with a mallet or driver on a hit point 132 to cut through sediment and vegetation and seal off the extracted sample inside the sample extraction box 100.

The removable connector 140 includes a plurality of connecting members configured to be gripped or held to drive the extraction box 100 in the direction between the front side and the back side of the extraction box and to lift the extraction box 100 in the upward direction. In the example shown, the removable connector 140 includes two connecting links 142, 144 removably attached to the right side wall and the left side wall 120 near or adjacent the open top side of the extraction box 100. A plurality of attachment links or bars or beams 146, 148 are attached to and disposed above the two connecting links 142, 144. The attachment links 146, 148 are configured to be gripped or held to drive the extraction box 100 in the direction between the front side and the back side of the extraction box and to lift the extraction box in the upward direction. The removable connector 140 further includes a first extension plate 150 and a second extension plate 152 attached to the two connecting links or bars or beams 142, 144 and extending above the extraction box 100. The plurality of attachment links 146, 148 are attached to the first extension plate 150 and the second extension plate 152. The two connecting links 142, 144 each have one end attached to the right side wall 120 and another end attached to the left side wall 120. Two attachment links 146, 148 each have one end attached to the first extension plate 150 and another end attached to the second extension plate 152. The two connecting links 142, 144 may be parallel connecting links. The two attachment links 146, 148 are parallel attachment links.

This embodiment provides a quick-connect configuration of the removable connector 140 with the attachment links 146, 148 for engaging the heavy machinery with the extraction box 100 to drive it into a dune and lift it out of the dune. It is merely illustrative and not limiting. In other embodiments, the quick-connect removable connector 140 may have other configurations.

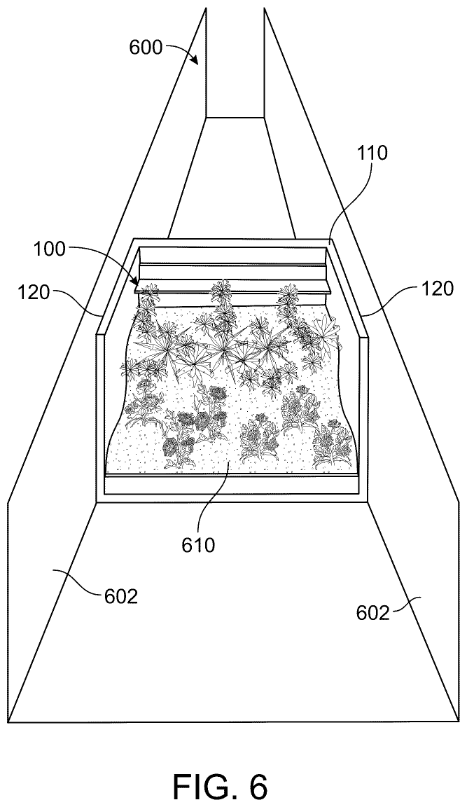

FIG. 6 shows the extraction box 100 disposed in a flume 600 for testing the extracted dune sample 610 with vegetation intact. After lifting and transporting the extraction box 100 from the dune, it is lowered into a flume 600 having two side walls 602 extending in a longitudinal direction. The extraction box 100 is placed in the flume to position the right side wall 120 of the extraction box next to one side wall 602 of the flume and the left side wall 120 of the extraction box next to another side wall 602 of the flume 600. Either the front gate 110 or the back plate 112 can be removed to provide an open front side or an open back side to allow wave propagation into the box 100, or both can be removed to allow wave overtopping through the box 100, for testing the dune sample 610. The excavator connector 140 can be removed to allow full profile view of the sample for measurements during testing. In one embodiment, the box 100 has a height of about 3′, a length of about 4′, and a width of about 2.5′. The pair of side panels or walls 120 are separated by the width. The metal may be steel.

The sample extraction box 100 allows for the reconfiguration of the sampler 100 to transition smoothly from a contained unit, for testing purposes. The dune sampler 100 is configured to allow for direct testing within the sampler 100 and does not require the removal of the sample 610 from the sampler 100. This design facilitates ease and efficiency of testing and reduces the risk of altering the sample 610 before testing.

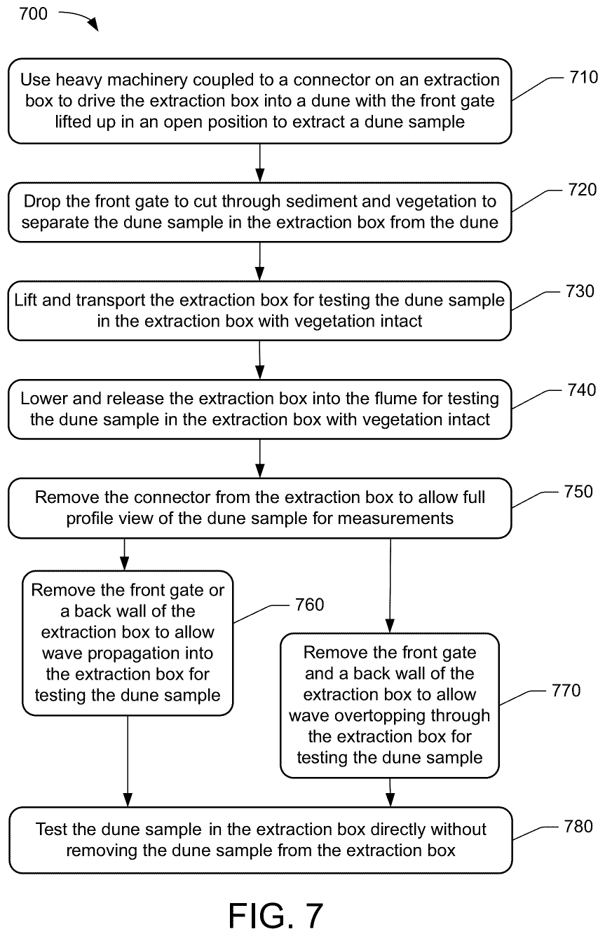

FIG. 7 is a flow diagram illustrating an example of a dune segment extraction and testing process. In step 710, heavy machinery (e.g., excavator or backhoe) is coupled to a removable connector 140 of an extraction box 100 to drive the extraction box 100 into a dune with the front gate 110 lifted up in an open position to extract a dune sample 610. In step 720, the front gate 110 is dropped to cut through sediment and vegetation to separate and seal off the dune sample 610 in the extraction box 100 from the dune. In step 730, the extraction box 100 is lifted and transported for testing the dune sample 610 in the extraction box with vegetation intact. In step 740, the extraction box is lowered and released into the flume 600 for testing the dune sample 610 in the extraction box 100 with vegetation intact. In step 750, the connector 140 is removed from the extraction box to allow full profile view of the dune sample 610 for measurements during testing. Two example options are presented. In step 760, one of the front gate 110 and the back wall 112 of the extraction box 100 is removed to allow wave propagation into the extraction box 100 for testing the dune sample 610. Alternatively, in step 770, both the front gate 110 and the back wall 112 are removed to allow wave overtopping through the extraction box 100 for testing the dune sample 610. In step 780, the dune sample 610 is tested in the extraction box 100 directly without removing the dune sample 610 from the extraction box 100.

### 3. Evaluating Impact of Belowground Biomass to Dune Stability Under Extreme Wave Events

The extraction box 100 can be used to evaluate the impact of belowground biomass to dune stability under extreme wave events. Testing of the extracted dune sample with natural vegetation intact can be used for investigating dune stability to inform restoration design and management guidance.

Established dunes are very complex systems and the role of vegetation on their stability from an engineering perspective is not yet fully understood. Laboratory studies have continually investigated the role of vegetation on dune erosion and stability; however, these studies have noted limitations given they often mimic vegetation and are not true representations of real-world vegetation. These limitations have been circumvented by utilizing a real-word dune section, in a controlled, laboratory environment. For instance, a 5-foot wide (in the longshore direction) dune segment has been cut from the beach and transported to flume facilities. A series of hydrodynamic conditions are evaluated with the dune segment in a laboratory setting. In doing this, a controlled environment is created and used to evaluate established dune behavior under wave events, without the need for complex field data that often cannot be taken during extreme events. Results from this experiment are used to help in the validation of the morphology numerical modeling routines under development.

In short, this work extracts a dune segment from field site and implement a controlled laboratory experiment to investigate the physical role of ecological components on dune stability.

Prior to this invention, the stability of a naturally developed dune has not been previously evaluated within a controlled environment. Prior laboratory experiments have included mimic vegetation, lab-grown vegetation, and transplanted dune plants within constructed dunes, but not a dune grown in its native environment. This dune sampler invention has allowed for the first-ever, in-tact dune sample to be taken directly from its native environment, transported, and tested in a wave flume. The successful completion of this testing has provided confirmation that native vegetated dunes provide significant protection from wave action, as compared to a non-vegetated constructed dune.

This study will lead to an improved understanding of dune establishment after planting and dune erosion following storms. This knowledge is critically needed to support more climate-ready and climate-resilient dune management in the years to come as the nation's beaches face rising sea levels and more intensive storms. The restoration experiments and surveys of dunes with different vegetation cover and restoration histories have been intentionally designed to generate knowledge that will improve future dune restoration efforts. The field components will additionally provide the observations needed to parameterize the effects of belowground vegetation. These parameterizations will promote a better estimation of the dune erosion rates during extreme storms. The physical and idealized models of dune erosion will improve dune management practices and help identify locations where severe erosion and/or dune breaches are likely.

This project advances knowledge that will support the management of coastal dunes that are more storm resilient and faster to recover after storm events. In particular, one will be testing the hypothesis that dunes with diverse and deep-rooted vegetation will be less likely to erode and breach during storms, and therefore will achieve cost savings over time by being less likely to require further maintenance. The modeling aspects of this project will also support coastal managers in their efforts to identify locations where enhancing vegetation may achieve high returns on investment in helping stabilize dunes and maintaining their ecosystem services.

Embodiments of the invention can be manifest in the form of methods and apparatuses for practicing those methods. Traditional physical models utilize mimic vegetation or transplanted vegetation grown for a limited amount of time but are not representative of a naturally established system. Such an approach does not measure ecological components within a natural system that may contribute to dune stability. As compared to the traditional models, the benefits of implementing this technology include smooth transition of the dune sampler in the form of a transition box from an extraction unit to extract a dune sample to a test unit for testing the dune sample without removing the dune sample from the extraction box. This facilitates easy and efficient testing of the dune sample and reduces the risk of altering the sample before testing.

The inventive concepts taught by way of the examples discussed above are amenable to modification, rearrangement, and embodiment in several ways. Accordingly, although the present disclosure has been described with reference to specific embodiments and examples, persons skilled in the art will recognize that changes may be made in form and detail without departing from the spirit and scope of the disclosure.

An interpretation under 35 U.S.C. § 112(f) is desired only where this description and/or the claims use specific terminology historically recognized to invoke the benefit of interpretation, such as “means,” and the structure corresponding to a recited function, to include the equivalents thereof, as permitted to the fullest extent of the law and this written description, may include the disclosure, the accompanying claims, and the drawings, as they would be understood by one of skill in the art.

To the extent the subject matter has been described in language specific to structural features and/or methodological steps, it is to be understood that the subject matter defined in the appended claims is not necessarily limited to the specific features or steps described. Rather, the specific features and steps are disclosed as example forms of implementing the claimed subject matter. To the extent headings are used, they are provided for the convenience of the reader and are not to be taken as limiting or restricting the systems, techniques, approaches, methods, devices to those appearing in any section. Rather, the teachings and disclosures herein can be combined, rearranged, with other portions of this disclosure and the knowledge of one of ordinary skill in the art. It is the intention of this disclosure to encompass and include such variation.

The indication of any elements or steps as “optional” does not indicate that all other or any other elements or steps are mandatory. The claims define the invention and form part of the specification. Limitations from the written description are not to be read into the claims.

Unless explicitly stated otherwise, each numerical value and range should be interpreted as being approximate as if the word “about” or “approximately” preceded the value or range.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

What is claimed is:

1. An extraction box comprising:
a bottom;
a right side wall connected to the bottom;
a left side wall connected to the bottom;
a first wall connected to the bottom, the right side wall, and the left side wall, to form a back wall on a back side of the extraction box;
a second wall connected to the bottom, the right side wall, and the left side wall, to form a front wall on a front side of the extraction box, the second wall movable between a closed position to enclose the front side of the extraction box and an open position to open up the front side of the extraction box; and
a removable connector removably attached to the right side wall and the left side wall to be disposed adjacent a top side of the extraction box, to be driven in a direction between the front side and the back side of the extraction box and to be lifted in an upward direction, the removable connector being removable from the extraction box to provide an open top side of the extraction box.

2. The extraction box of claim 1,
wherein the second wall comprises a drop gate slidable along vertical tracks between the open position and the closed position and configured to drop from the open position to the closed position by releasing the drop gate from the vertical tracks and hitting a hit point on the drop gate.

3. The extraction box of claim 1,
wherein the first wall is removable from the extraction box to provide/expose an open back side of the extraction box.

4. The extraction box of claim 1,
wherein the removable connector comprises a plurality of connecting members configured to be gripped to drive the extraction box in the direction between the front side and the back side of the extraction box and to lift the extraction box in the upward direction.

5. The extraction box of claim 1, wherein the removable connector comprises:
two connecting links removably attached to the right side wall and the left side wall adjacent the top side of the extraction box; and
a plurality of attachment links attached to and disposed above the two connecting links, the attachment links configured to be gripped to drive the extraction box in the direction between the front side and the back side of the extraction box and to lift the extraction box in the upward direction.

6. The extraction box of claim 5, wherein the removable connector further comprises:
a first extension plate and a second extension plate attached to the two connecting links and extending above the extraction box;
wherein the plurality of attachment links are attached to the first extension plate and the second extension plate.

7. The extraction box of claim 6,
wherein the two connecting links each have one end attached to the right side wall and another end attached to the left side wall; and
wherein the plurality of attachment links comprise two attachment links each having each have one end attached to the first extension plate and another end attached to the second extension plate.

8. The extraction box of claim 7,
wherein the two connecting links are parallel connecting links; and
wherein the two attachment links are parallel attachment links.

9. A dune sample extraction method, comprising:
holding an extraction box having a bottom; a right side wall connected to the bottom; a left side wall connected to the bottom; a first wall connected to the bottom, the right side wall, and the left side wall, to form a back wall on a back side of the extraction box; a second wall connected to the bottom, the right side wall, and the left side wall, to form a front wall on a front side of the extraction box; by a removable connector removably attached to the right side wall and the left side wall to be disposed adjacent a top side of the extraction box, the second wall being disposed in an open position to open up the front side of the extraction box;
driving the open front side of the extraction box, through the removable connector, into a dune to collect a dune sample;
moving the second wall from the open position to a closed position to cut through sediment and vegetation to separate the dune sample in the extraction box from the dune;
lifting the extraction box from the dune by the removable connector;
transporting the extraction box to a flume having two side walls extending in a longitudinal direction;
lowering the extraction box into the flume to position the right side wall of the extraction box next to one side wall of the flume and the left side wall of the extraction box next to another side wall of the flume; and
removing at least one of the first wall or the second wall from the extraction box to allow wave propagation into the extraction box for testing the dune sample.

10. The dune sample extraction method of claim 9, further comprising:
sliding the second wall to the open position to open up the front side of the extraction box before driving the open front side of the extraction box into the dune to collect the dune sample.

11. The dune sample extraction method of claim 9,
wherein holding the extraction box by the removable connector comprises gripping a plurality of connecting members of the removable connector to drive the extraction box in a direction between the front side and the back side of the extraction box; and
wherein lifting the extraction box from the dune by the removable connector comprises gripping the plurality of connecting members of the removable connector to lift the extraction box in an upward direction.

12. The dune sample extraction method of claim 9,
wherein holding the extraction box by the removable connector comprises gripping a plurality of attachment links attached to and disposed above two connecting links removably attached to the right side wall and the left side wall adjacent the top side of the extraction box to drive the extraction box in a direction between the front side and the back side of the extraction box and to lift the extraction box in an upward direction.

13. The dune sample extraction method of claim 9,
wherein holding the extraction box by the removable connector comprises gripping a plurality of attachment links attached to a first extension plate and a second extension plate which are attached to two connecting links and extending above the extraction box, the two connecting links removably attached to the right side wall and the left side wall adjacent the top side of the extraction box to drive the extraction box in a direction between the front side and the back side of the extraction box and to lift the extraction box in an upward direction.

14. The dune sample extraction method of claim 9, further comprising:
removing the removable connector from the extraction box to provide an open top side of the extraction box disposed in the flume.

15. The dune sample extraction method of claim 9, further comprising:
removing the first wall from the extraction box disposed in the flume to provide an open back side of the extraction box to allow wave propagation into the extraction box for testing the dune sample.

16. The dune sample extraction method of claim 15, further comprising:
removing the second wall from the extraction box to allow wave overtopping through the extraction box for testing the dune sample.

17. A dune sample extraction method, comprising:
holding an extraction box having a bottom; a right side wall connected to the bottom; a left side wall connected to the bottom; a first wall connected to the bottom, the right side wall, and the left side wall, to form a back wall on a back side of the extraction box; a second wall connected to the bottom, the right side wall, and the left side wall, to form a front wall on a front side of the extraction box; by a removable connector removably attached to the right side wall and the left side wall to be disposed adjacent a top side of the extraction box, the second wall being disposed in an open position to open up the front side of the extraction box;
driving the open front side of the extraction box, through the removable connector, into a dune to collect a dune sample;
moving the second wall from the open position to a closed position to cut through sediment and vegetation to separate the dune sample in the extraction box from the dune;
lifting the extraction box from the dune; and
performing test on the dune sample in the extraction box without removing the dune sample from the extraction box.

18. The dune sample extraction method of claim 17, wherein performing test on the dune sample comprises:
transporting the extraction box to a flume having two side walls extending in a longitudinal direction;
lowering the extraction box into the flume to position the right side wall of the extraction box next to one side wall of the flume and the left side wall of the extraction box next to another side wall of the flume; and
removing at least one of the first wall or the second wall from the extraction box to allow wave propagation into the extraction box for testing the dune sample.

19. The dune sample extraction method of claim 18, further comprising:
removing the first wall and the second wall from the extraction box to allow wave overtopping through the extraction box for testing the dune sample.

20. The dune sample extraction method of claim 18, further comprising
removing the removable connector from the extraction box in the flume to provide an open top side of the extraction box disposed in the flume.
